(19) United States
(12) Patent Application Publication
(10) Pub. No.: US 2026/0134537 A1
(43) Pub. Date: May 14, 2026

(54) SYSTEMS AND METHOD FOR STANDARDIZED VIDEO CAPTURE AND AI-BASED ANALYSIS OF SMALL-OBJECT HAND TASKS FOR DIAGNOSIS OF CERVICAL MYELOPATHY

(71) Applicant: University of Virginia Patent Foundation, Charlottesville, VA (US)

(72) Inventors: Xudong Li, Charlottesville, VA (US); Ke Wang, Charlottesville, VA (US)

(21) Appl. No.: 19/387,984

(22) Filed: Nov. 13, 2025

Related U.S. Application Data
(60) Provisional application No. 63/719,914, filed on Nov. 13, 2024.
(60) Provisional application No. 63/908,891, filed on Oct. 31, 2025.

(51) Int. Cl. G06T 7/00; A61B 5/00; G06T 7/73; G06V 10/62; G06V 10/774; G06V 20/40; G06V 40/10; G06V 40/20

(52) U.S. Cl. CPC G06T 7/0012; A61B 5/407; G06T 7/73; G06V 10/62; G06V 10/774; G06V 20/46; G06V 40/107; G06V 40/28; G06T 2207/10016; G06T 2207/20081; G06T 2207/30196; G06T 2207/30204; G06V 2201/03

(57) ABSTRACT

In various embodiments, a method comprises: capturing, by computing hardware, video; extracting, by the computing hardware, one or more per-frame pose features from the video; extracting, by the computing hardware, one or more per-video temporal features from the video; causing, by the computing hardware, at least one of a machine-learning model or a rules-based mode to generate at least one of a risk level prediction or a severity score based on the one or more per-frame pose features and the one or more per-video temporal features, the risk level prediction and the severity score being related to a potential diagnosis of a medical condition; producing, by the computing hardware, an output based on at least one of the risk level prediction or the severity score; and generating, using a machine learning model, a treatment recommendation for the condition.

### CROSS-REFERENCE TO RELATED APPLICATION

This application claims the benefit of U.S. Provisional Patent Application Ser. No., 63/719,914, filed Nov. 13, 2024 and U.S. Provisional Patent Application Ser. No., 63/908,891, filed Oct. 31, 2025, the disclosures of which are hereby incorporated herein by reference in their entirety.

### TECHNICAL FIELD

The disclosure provided herein relates to artificial intelligence type computers and digital data processing systems and corresponding data processing methods and products for analyzing video and emulating intelligence to derive diagnoses and generate recommendations for treatment.

### BACKGROUND

A significant technical challenge encountered in the context of providing efficient functioning of a computer is analyzing video to derive diagnoses based on the video and generate recommendations for treatment based on those diagnoses. Accordingly, various embodiments of the disclosure provided herein provide improvements in computer functionality and

### SUMMARY

In general, various aspects of the present invention provide methods, apparatuses, systems, computing devices, computing entities, and/or the like for diagnosing and generating treatment recommendations for a medical condition.

In some embodiments, a method comprises: capturing, by computing hardware, video; extracting, by the computing hardware, one or more per-frame pose features from the video; extracting, by the computing hardware, one or more per-video temporal features from the video; causing, by the computing hardware, at least one of a machine-learning model or a rules-based mode to generate at least one of a risk level prediction or a severity score based on the one or more per-frame pose features and the one or more per-video temporal features, the risk level prediction and the severity score being related to a potential diagnosis of a medical condition; producing, by the computing hardware, an output based on at least one of the risk level prediction or the severity score; configuring, by the computing hardware, a graphical user interface based on the output; and providing, by the computing hardware, the graphical user interface for display on a computing device.

In some embodiments, the video comprises video of an object transfer task; the one or more per-frame pose features comprise one or more hand or arm landmarks for each frame of the video; and extracting the one or more per-video temporal features comprises aggregating a sequence of the one or more per-frame pose features to derive at least one of a spectral stability index during performance of the object transfer task, a stack placement precision index for the object transfer task, or a completion time for the object transfer task. In some embodiments, the stack placement precision index defines a measure of how accurately each object in the object transfer task is placed relative to a second object. In particular embodiments, the spectral stability index provides an indication of a stability of at least a portion of a body of an individual performing the object transfer task during performance of the object transfer task.

In various aspects, the output comprises a diagnosis with respect to the medical condition. In some aspects, capturing the video comprises at least one of: calibrating, by the computing hardware, an imaging device against a set of video capture constraints; redacting, by the computing hardware, at least one piece of identifying information from the video; or storing, by the computing hardware, metadata in association with the video, the metadata including at least one of a device ID of the imaging device, one or more calibration parameters of the imaging device, or a configuration hash of the imaging device. In a particular embodiment, the medical condition is cervical myelopathy.

A system, in some aspects, comprising: a non-transitory computer-readable medium storing instructions; and a processing device communicatively coupled to the non-transitory computer-readable medium. In some embodiments, the processing device is configured to execute the instructions and thereby perform operations comprising: preparing a set of training data, the set of training data comprising a set of videos and each video in the set of videos having a respective set of labels and including a respective object transfer task; extracting, from each frame in each video in the set of videos, at least one respective per-frame pose feature; extracting, from each video in the set of videos, at least one respective per-video temporal feature; training, for a first task of generating at least one of a risk level prediction or a severity score for use in diagnosing a condition or recommending a treatment for the condition, a machine-learning model using each respective set of labels, the at least one respective feature pose for each frame in each video, and each at least one respective per-video temporal feature; and providing the machine-learning model for use in performance of the first task.

In some embodiments, the at least one respective feature pose for each frame in each video and each at least one respective per-video temporal feature comprise a set of video-derived features; and the operations further comprise: extracting at least one non-video feature for each respective video in the set of videos, the at least one non-video feature defining at least one of a respective demographic of a respective subject in each respective video, a respective piece of clinical data for each respective subject, or respective structured input for each respective subject. ; and fusing the at least one non-video feature with the set of video-derived features prior to training the machine-learning model.

In some embodiments, at least one respective label in each respective set of labels comprises one or more of: one or more calibration parameters for the respective video; a device ID used to capture each respective video; or one or more clinical labels for a subject present in each respective video. In various embodiments, the one or more clinical labels comprise at least one of an age of the subject, a sex of the subject, a diagnosis for the subject, or symptom duration data for the subject.

In particular aspects, extracting the at least one respective per-video temporal feature comprises aggregating a sequence of the at least one respective per-frame pose feature for each respective video to derive at least one of a respective spectral stability index during performance of the object transfer task, a respective stack placement precision index for the respective object transfer task, or a completion time for the respective object transfer task; the respective spectral stability index during performance of the object transfer task defines a tremor-free stability of a respective individual performing the respective object transfer task during performance or the object transfer task; and the respective stack placement precision index for the respective object transfer task defines a measure of how accurately the respective individual places a first object during performance of the object transfer task with respect to at least one of a second object or a fiducial marker.

In some aspects, the operations further comprise providing at least one of the respective spectral stability index during performance of the object transfer task, the respective stack placement precision index for the respective object transfer task, or the completion time for the respective object transfer task as training data for the machine-learning model. In other aspects, the operations further comprise: receiving new video of an individual; extracting one or more individual-specific per-frame pose features from the new video; extracting one or more individual-specific per-video temporal features from the new video; causing a machine-learning model to generate at least one of an individual-specific risk level prediction or an individual-specific severity score based on the individual-specific one or more per-frame pose features and individual-specific the one or more per-video temporal features; producing an output from the machine-learning model based on at least one of the individual-specific risk level prediction or the individual-specific severity score, the output comprising at least one of an individual-specific condition diagnosis or an individual-specific treatment recommendation; configuring, by the computing hardware, a graphical user interface based on the output; and providing, by the computing hardware, the graphical user interface for display on a computing device.

A method, in some embodiments, comprises: receiving, by computing hardware, a first video at a first time; extracting, by the computing hardware, one or more first per-frame pose features from the first video; extracting, by the computing hardware, one or more first per-video temporal features from the first video; causing, by the computing hardware, at least one of a first machine-learning model or a first rules-based mode to generate at least one of a first risk level prediction or a first severity score based on the one or more first per-frame pose features and the one or more first per-video temporal features, the first risk level prediction or the first severity score being related to a potential diagnosis of a medical condition; causing, by the computing hardware, at least one of a second machine-learning model or a second rules-based model to generate a treatment recommendation based on the at least one of the first risk level prediction or the first severity score; receiving, by computing hardware, a second video at a second time subsequent to execution of at least a portion of a treatment plan indicated by the treatment recommendation; extracting, by the computing hardware, one or more second per-frame pose features from the second video; extracting, by the computing hardware, one or more second per-video temporal features from the second video; causing, by the computing hardware, at least one of the first machine-learning model or the first rules-based mode to generate at least one of a second risk level prediction or a second severity score based on the one or more second per-frame pose features and the one or more second per-video temporal features; generating a comparison of at least one of the first risk level prediction or the second severity score with the second risk level prediction or the second severity score based; configuring, by the computing hardware a graphical user interface with an indication of the comparison; and providing, by the computing hardware, the graphical user interface for display on a computing device.

In some embodiments, the method further comprises providing, by the computing hardware, the comparison and the treatment plan as training data to at least one of the second machine-learning model or the rules-based model. In some embodiments, the first machine-learning model is the second machine-learning model; and the first rules-based model is the second rules-based model.

### BRIEF DESCRIPTION OF THE DRAWINGS

In the course of this description, reference will be made to the accompanying drawings, which are not necessarily drawn to scale, and wherein:

FIG. 1 depicts an example of a computing environment that can be used for diagnosing and generating treatment plans for particular conditions based on object transfer task analysis according to various aspects;

FIG. 2 depicts an example of a process for capturing video and other imaging for use in diagnosing and generating treatment plans for particular conditions in accordance with various aspects of the present disclosure;

FIG. 3 depicts an example of an object transfer task test sheet in accordance with various aspects of the present disclosure;

FIG. 4 depicts another example of an object transfer task test sheet in accordance with various aspects of the present disclosure;

FIG. 5 depicts an example of a performance of an object transfer task in accordance with various aspects of the present disclosure;

FIG. 6 depicts an example of a process for training a machine-learning model for use in diagnosing and generating treatment plans for particular conditions based on video data in accordance with various aspects of the present disclosure;

FIG. 7 depicts an example of a process for analyzing video for use in diagnosing particular conditions in accordance with various aspects of the present disclosure;

FIG. 8 depicts an example of a process for generating treatment recommendations in accordance with various aspects of the present disclosure;

FIG. 9 depicts an example of a process for tracking changes in condition severity in accordance with various aspects of the present disclosure;

FIG. 10 depicts an example of a process for training a machine-learning model for use in generating treatment plans and/or recommendations in accordance with various aspects of the present disclosure;

FIG. 11 depicts an example of a system architecture that may be used in accordance with various aspects of the present disclosure; and

FIG. 12 depicts an example of a computing entity that may be used in accordance with various aspects of the present disclosure.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the disclosure. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method and/or process can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an,” and “the” include plural referents unless the context clearly dictates otherwise. Thus, for example, reference to “a spacer,” “a guide nucleic acid,” or “an miRNA,” including, but not limited to, mixtures or combinations of two or more such spacers, guide nucleic acids, or miRNAs, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Overview

As noted above, a significant technical challenge encountered in the context of providing efficient functioning of a computer is analyzing video to derive diagnoses based on the video and generate recommendations for treatment based on those diagnoses. For example, Cervical spondylotic myelopathy (CSM) stands as a significant global health concern, representing a primary cause of non-traumatic spinal cord dysfunction. Early diagnosis and intervention are essential to prevent permanent spinal cord damage and improve outcomes. However, diagnosing CSM is challenging due to its insidious onset and nonspecific nature of clinical symptoms.

For example, CSM diagnosis often relies on imprecise visual observations of a patient. Conventional techniques for diagnosing CSM and other conditions requires subjective determinations applied to imprecise visual observations, such as attempting to glean subtle differences in movement between different patients, identifying particular movements or change in movement that may differ for different patients suffering the same condition, attempting to visually perceive multiple different indicating factors on multiple portions of a patients'body simultaneously, manually tracking particular patient progress against various metrics, performing on-the-fly analysis of patient performance that is not possible in the human mind, etc. Thus, aspects described herein improve computer-implemented processes that are unique to analyzing video containing patient movement during performance of certain activities to generate diagnoses and potential treatments that may provide the most optimal outcomes for those patients, thereby providing a more suitable solution for automating and improving tasks previously performed by humans.

Particular aspects described herein provide improved patient analysis through the use of a particular physical test, for which video is captured and analyzed. As may be appreciated by one skilled in the art, variations of such a physical test may be suitable for use in diagnosing conditions described herein. In some embodiments, the use of the same set of tasks in the particular physical test may provide an opportunity to leverage improved computing processes (e.g., through machine learning) for use in analyzing video of such tasks.

In one example described herein, a simple test “Coin Test” (or object transfer test) provides high sensitivity and high specificity for detecting hand motor and sensory dysfunction. In some examples, the test involves patients stacking five pennies from one pile to another. While unaffected individuals typically perform with ease, CSM patients often encounter significant challenges, highlighting impairments in fine motor movements and sensory functions. This Coin Test (or small object test) may, among other things, transform the early diagnosis and management of CSM by providing an accessible, reproducible, and comprehensive tool for identifying both motor and sensory dysfunction. By facilitating timely intervention, it has the potential to improve patient outcomes and prevent irreversible long-term disability.

Furthermore, it can be technically difficult to provide accurate predictions as to a risk level of lack of intervention for a particular patient or ascertain a severity of a patient exhibiting certain physical characteristics during performance of an object transfer test or other test. In particular, there are significant technical challenges related to generating risk level predictions or severity scores for a particular patient that account for particular attributes of the patient themself, the performance of the patient during the object transfer test, and the like. Conventional risk predictions and/or severity score determinations rely on generic, non-personalized data and other results that are not specific to the patient and their performance, because such predictions may not take into account the unique characteristics of the patient themself, diagnoses of similarly-situated other patients, and other unique factors and attributes related to the patient, other patients, or performance of the task. It can further be technically difficult to identify the most effective potential treatment plan or recommendation for any specific individual patient.

Accordingly, various aspects of the present disclosure overcome many of the technical challenges mentioned above associated with diagnosing and generating treatment plans for particular conditions based on object transfer task analysis. In particular, various aspects of the present disclosure provide improved methods for generating impact predictions by identifying similar, previously completed campaigns and prior performance data in prior campaigns for each participant in the set of potential current participants and using that data to determine more accurate predictions with respect to a current potential or planned campaign. Additionally, various aspects of the present disclosure provide improved methods for identifying similar prior campaigns to provide more accurate impact predictions. In this way, when determining which particular potential new campaigns to initiate, the system may enable more optimal selection of new campaigns to which to assign a limited set of resources. In this way, the system is configured to increase a likelihood of greater impact across a set of projects form a limited set of resources.

### Example Computing Environment

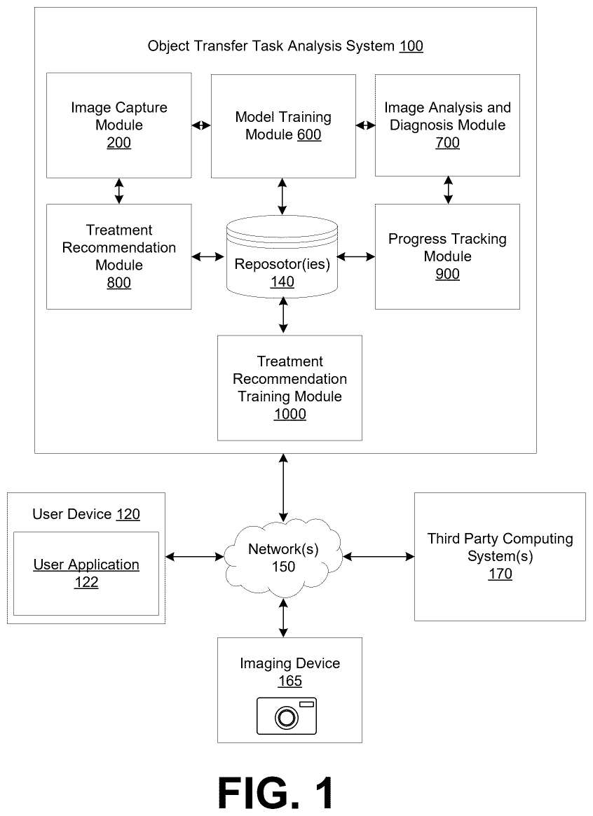

FIG. 1 depicts an example of a computing environment that can be used for diagnosing and generating treatment plans for particular conditions based on object transfer task analysis according to various aspects. For example, users may use a computing system to capture video of a patient or other individual performing a task (e.g., object transfer task). The computing environment may then be used to analyze the video using one or more machine learning models to generate risk predictions, assign severity scores, diagnose conditions, generate treatment recommendations, and the like.

In various aspects, an object transfer task analysis system 100 is provided within the computing environment that includes software components and/or hardware components to aid users in capturing video of object transfer tasks, training machine learning models for use in analyzing the video, deploying the machine learning models, and the like. For instance, the object transfer task analysis system 100 may provide access an object transfer task analysis platform that is accessible over one or more networks 150 (e.g., the Internet) by a user accessing a user application 122 on a user computing device 120.

Here, the object transfer task analysis system 100 may provide the user computing device 120 with one or more graphical user interfaces (e.g., webpages, software applications, etc.) through the service to access the object transfer task analysis system 100. The user may use the service in performing functionality associated with video capture and analysis.

In addition to the graphical user interfaces, the object transfer task analysis system 100may include one or more interfaces (e.g., application programming interfaces (APIs)) for communicating and/or accessing third party computing system(s) 170 over the network(s) 150. For instance, the object transfer task analysis system 100 may access a third party computing system 170 via one of the interfaces to access patient information, perform one or more computing steps described herein, and the like. In still other examples, the object transfer task analysis system 100 may access the third party computing system 170 to cause the third party computing system 170 to perform one or more video analysis functions on the video (e.g., feature extraction), and the like.

In some instances, the object transfer task analysis system 100 may include one or more repositories 140 that can be used for storing data related to users/patients, model training data, and other data. In other aspects, the one or more repositories 140 may store data related to potential treatment options, patient progress data, or other suitable data.

In some aspects, the object transfer task analysis system 100 executes an image capture module 200 to capture video or other imaging data for use in diagnosing and generating treatment plans for particular conditions. In some aspects, the image capture module 200 may provide for controlled capture (e.g., in terms of angle, distance, illumination, frame rate, background, and the like) to provide greater reproducibility and cross-site comparability of video during downstream analysis.

In some other aspects, the object transfer task analysis system 100 executes a model training module 600 for training a machine-learning model for use in diagnosing and generating treatment plans for particular conditions based on video data. In various aspects, the object transfer task analysis system 100 prepares training data, performs feature extraction, and uses the training data to train and calibrate

In additional or alternative aspects, the object transfer task analysis system 100 executes an image analysis and diagnosis module 700. In some embodiments, the image analysis and diagnosis module 700 is configured for analyzing video for use in diagnosing particular conditions in accordance with various aspects of the present disclosure. In some embodiments, the image analysis and diagnosis module 700 causes one or more machine learning models to generate a risk level predication and/or severity score for a patient with respect to a medical condition from video.

In additional or alternative aspects, the object transfer task analysis system 100 executes a treatment recommendation module 800 configured for generating treatment recommendations in accordance with various aspects of the present disclosure. For example, the treatment recommendation module 800 may be configured to process patient data using a machine-learning model trained on past courses of treatment for other patients to generate a recommended treatment or course of treatment.

In additional or alternative aspects, the object transfer task analysis system 100 executes a progress tracking module 900 for tracking changes in condition severity for a particular patient over time. In additional or alternative aspects, the object transfer task analysis system 100 executes a treatment recommendation training module 1000. The treatment recommendation training module 1000 may be configured for training a machine-learning model for use in generating treatment plans and/or recommendations.

Further detail is provided below regarding the configuration and functionality of the image capture module 200, model training module 600, image analysis and diagnosis module 700, treatment recommendation module 800, progress tracking module 900, and treatment recommendation training module 1000, according to various aspects of the disclosure.

It should be understood that various aspects of this disclosure refer to diagnosing particular medical conditions and the like. In various aspects, any such reference should be understood to encompass any suitable medical condition for which a potential diagnosis, severity score, and the like may be derived based on video taken of a patient.

The number of devices depicted in FIG. 1 are provided for illustrative purposes. In some aspects, different number of devices may be used. In various aspects, for example, while certain devices or systems are shown as single devices in FIG. 1, multiple devices may instead be used to implement these devices or systems.

In some aspects, the object transfer task analysis system 100 can include one or more third-party devices such as, for example, one or more servers operating in a distributed manner. The object transfer task analysis system 100 can include any computing device or group of computing devices, and/or one or more server devices.

Although the data repository 140 is shown as a single component, these components 140 may include, in other aspects, a single server and/or repository, servers and/or repositories, one or more cloud-based servers and/or repositories, or any other suitable configuration.

### Image Capture Module

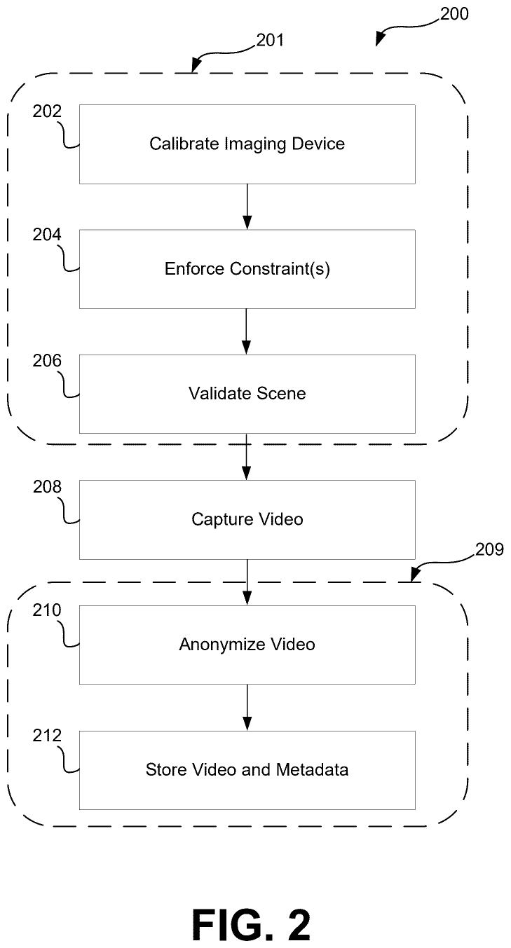

Turning now to FIG. 2, additional details are provided regarding an image capture module 200 for capturing video and other imaging data for use in diagnosing and generating treatment plans for particular conditions. For instance, the flow diagram shown in FIG. 2 may correspond to operations executed by computing hardware found in object transfer task analysis system 100 as it executes the image capture module 200. In particular embodiments, the image capture module 200 may include an initial set of pre-processing steps 201 configured to calibrate and otherwise prepare the imaging device for capturing the video required to perform the analysis/training described herein. The image capture module 200 may then include a video capture 208 step, followed by one or more post-processing steps 209.

At operation 202, the image capture module 200 calibrates an imaging device. The image capture module 200 may, for example, be configured to calibrate the imaging device to enable a controlled, reproducible, comparable video that is usable for training of and processing by a machine-learning module for the purposes discussed herein. In some embodiments, the image capture module 200 is configured to identify an object within a video with known geometry (e.g., a fiducial mat) to calibrate one or more imaging device settings, a position of the imaging device, and the like. For example, the system may be configured to calibrate the imaging device with respect to angle with respect to the object of known geometry, a distance from the object of known geometry, a lighting of the video, frame rate, background, and the like. The system may, for example, limit video capture until the device is properly calibrated to ensure consistent, reproducible capture conditions.

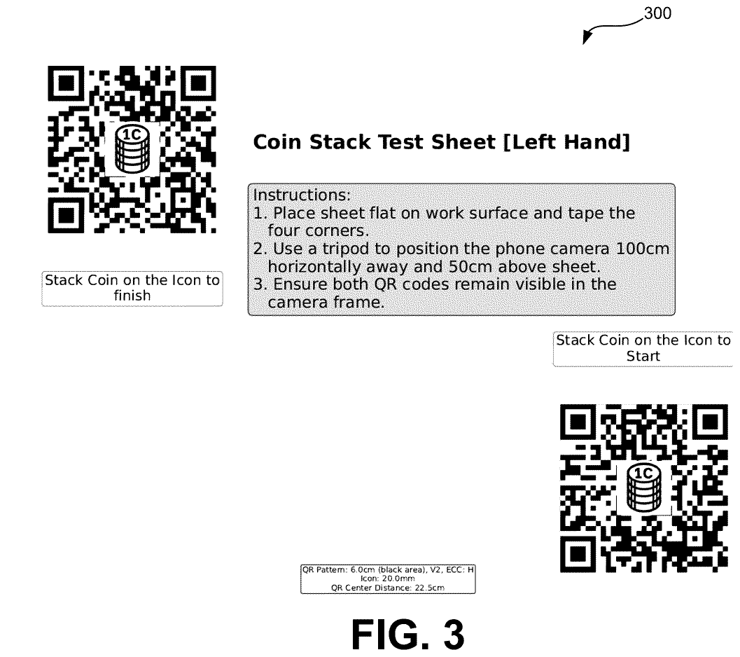

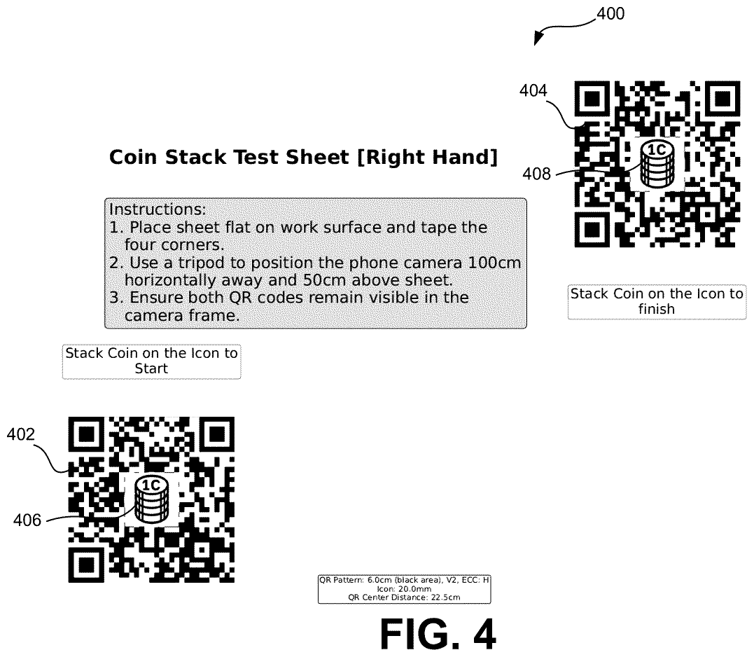

At operation 204, the image capture module 200 enforces particular capture constraints for the imaging device. This may include, for example, enforcing a camera pose constraint. The camera pos constraint may include a position of the camera with respect to the object of known geometry, such as a top-down pose constraint, a fixed angle constraint with respect to the object, etc. In still other examples, the system may enforce a distance constraint with respect to the camera and the object of known geometry (e.g., a distance of between about 35 cm and about 55 cm). In some embodiments, the system may infer the distance based on the geometry of one or more fiducials on the object. FIGS. 3 and 4 depict exemplary fiducial mats (e.g., objects of known geometry) which may be utilized in the context of the video capture of an object transfer task as described herein.

The system may further enforce capture constraints including a frame rate and/or exposure constraint. For example, in some embodiments, the system may enforce a specific frame rate requirement (e.g., at least about 30 frames per second), and the like. In still other examples, the system may enforce a resolution and/or stability constraint. In still other embodiments, the one or more constraints may include a brightness constraint (e.g., the scene is too dark or light).

At operation 206, the system may validate a capture scene prior to video capture. This may include, for example, detecting a surface plane (e.g., table), detecting a portion of a patient's body (e.g., hands), identifying one or more objects within frame (e.g., coins in embodiments that utilize a coin test), etc. In some embodiments, the system is configured to provide pass/fail validation prompts which may, for example, prompt a user of the imaging device to adjust settings, reposition the device, etc. In still other examples, the system may identify and confirm that a single hand is in frame (e.g., rather than both hands or a hand of another individual).

At operation 208, the system is configured to capture video, for example, using the imaging device 165. In various embodiments, the system is configured to capture the video during performance of the object transfer task. In some embodiments, the system is configured to enforce one or more capture constraints, or scene validation steps during the video capture. In this way, the system may automatically discard video for which constraints are deviated from during capture (e.g., when a second hand enters the scene, when lighting conditions change, when the fiducials or imaging device move or are moved, etc.). In other embodiments, the system is configured to prevent capture unless one or more constraints are in place. In this way, the system is configured to provide a consistent, reproducible capture process that provides better results when provided to the machine-learning model(s) for diagnostic and treatment prediction/recommendation purposes.

At operation 210, the system may be configured to anonymize video content, for example, by blurring and/or redacting a portion of the video. This may, for example, enable the system to use video data for machine-learning model training purposes while removing private data (e.g., identifying data) prior to us.

At operation 212, the system may be configured to store the captured imaging data (e.g., video) along with metadata that includes, for example, a device ID for the imaging device, one or more calibration parameters used to capture the data, a configuration hash, a timestamp of the capture, one or more quality control outcomes, and the like.

For illustrative purposes, the image capture module 200 is described with reference to implementations described above with respect to one or more examples described herein. Other implementations, however, are possible. In some aspects, the steps in FIG. 2 may be implemented in program code that is executed by one or more computing devices such as the object transfer task analysis system 100, the user device 120, or other system in FIG. 1. In some aspects, one or more operations shown in FIG. 2 may be omitted or performed in a different order. Similarly, additional operations not shown in FIG. 2 may be performed.

FIGS. 3 and 4 depict exemplary embodiment of an object transfer task instruction sheet. In the embodiment shown in FIG. 3, the coin stack test sheet 300 includes one or more fiducial markers and instructions for completion. Similarly, FIG. 4 depicts an exemplary coin stack test sheet 400 with instructions. In the example shown in FIG. 4, the sheet 400 includes a fiducial 402 in an initial position with a starting coin stack 406 as well as a fiducial 404 in a second position with a location of the final coin stack 408. As may be understood from these figures, when completing the coin test, a patient/individual places the sheet flat on a work surface, positions the imaging device and sets the imaging device up in accordance with the image capture module 200 described above, and moves a stack of coins from the first fiducial 402 to the second fiducial 404 in a stack.

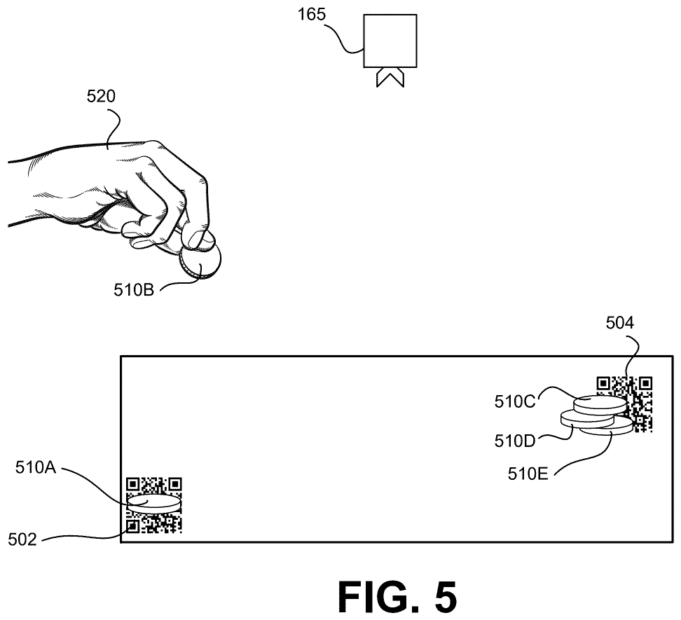

FIG. 5 depicts an example of an individual performing the coin test. As may be understood from this figure, the user may transfer a stack of coins 510A-E from the first fiducial marker 502 to the second fiducial marker 504 using their hand 520. As they perform the test, the system captures their performance using an imaging device 165.

This test requires a well-controlled coordination and directional movement of the arm and fingers. In this simple yet insightful assessment, patients are instructed to stack five penny coins on a flat surface—a seemingly straightforward task with profound diagnostic significance.

While unaffected individuals typically perform with ease, CSM patients often encounter significant challenges, highlighting impairments in fine motor movements and sensory functions. The elegance of the Coin Test lies in its ability to illuminate subtle difficulties experienced by individuals with CSM in a practical, clinically relevant manner. Such subtle difficulties may be impossible for a human to ascertain. As such, the system described herein provides solutions to the technical problems related to use of the test for diagnostic purposes in a way that is reproducible and provides consistent, trained diagnoses and treatment recommendations.

### Model Training Module

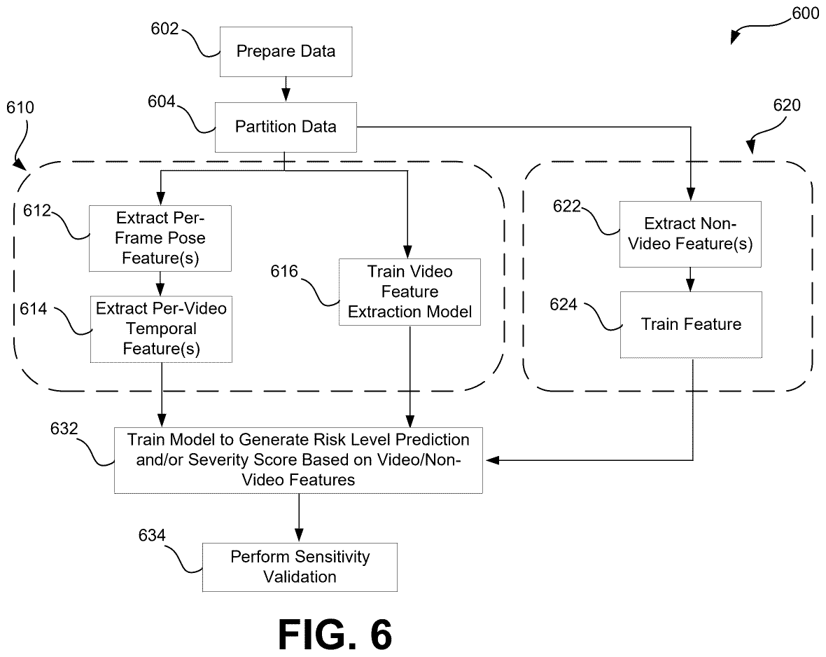

Turning now to FIG. 6, additional details are provided regarding a model training module 600 for training a machine-learning model for use in diagnosing and generating treatment plans for particular conditions based on video data. For instance, the flow diagram shown in FIG. 6 may correspond to operations executed by computing hardware found in the object transfer task analysis system 100 as it executes the model training module 600.

At operation 602, the model training module 600 prepares the data. For example, the system may reject any data that did not pass the image capture quality control checks described above with respect to the image capture module 200 described above. In some embodiment, each video is tagged with calibration parameters, device ID, and QC metadata. Clinical ground-truth labels (e.g., diagnosis, severity score, outcome) are verified by raters with inter-rater agreement recorded and tagged to the training data.

At operation 604, the system partitions the data into training, validation, and held-out test cohorts. The data is partitioned to maintain a balance across healthy patients and patients having a diagnosis to reduce bias during training of the machine-learning model. The system is configured to track and tag metadata to each piece of training video (e.g., site, device data, patient demographic data, and the like) to provide stratified sampling of the training data.

At operation set 610, the system extracts video features from the training data. This includes, at operation 612, extracting per-frame pose feature(s) from the video training data. From each video frame, for example, hand/arm landmarks may be extracted (e.g., via MediaPipe or equivalent). The system may compute derived kinematic features (velocity, acceleration, curvature, and the like) per frame. At operation 614, the system extracts per-video temporal feature(s). In some embodiments, sequences of per-frame features are aggregated into temporal descriptors using FFT/Wavelet transforms, PCA/autoencoders, or temporal encoders (e.g., transformers). The system may then embed quantitative features such as Spectral Stability Index (SSI), Stack Placement Precision (SPP), Completion time and related duration metrics.

SSI may provide a normalized, unitless index of tremor-free stability during quasi-static phases (e.g., grasp/placement). In one example, the system may compute SSI by rectifying a key point trajectory in canonical coordinates; band-pass 3-15 Hz to isolate micro-oscillation energy (E_band); compute total signal energy (E_total); and define SSI=1−(E_band/(E_total+ε)), clipped to [0,1]. In some embodiments, the higher the SSI, the lower the tremor energy, which may indicate higher stability.

SPP may provide a precision measure of how tightly each coin lands on the evolving stack centroid and orientation. For example, the system may computer SPP by, after placement: (1) fit coin ellipse→center c_i and angle θ_i; (2) compute normalized radial error e_{r,i}=∥c_i−ar c∥/R and angular error e_{θ, i}=|θ_i−ar θ| (normalized to [0,1]); (3) aggregate dispersion D=w_r·SD(e_r)+w_θ·SD(e_θ); define SPP=1−min(1, D) (or use exp(−D)). In some embodiments, the higher the SPP, the tighter, more consistent placements.

At operation 616, the system may train the video feature extraction model. This may, for example, provide an alternative training pathway than operations 612 and 614 and enable the system to provide training data to a third party computing system 170, for example, for the purposes of training video feature extraction for a video-to-feature single model.

At operation set 620, the system may extract non-video features. This may include, at operation 622, extract non-video features such as demographic, clinical, human input, or device metadata (e.g., age, sex, diagnosis, symptom duration, site, device type). Features may include scalar values (e.g., age), categorical encodings (e.g., sex, site), or structured inputs (e.g., baseline clinical scores). Human input can include completion rate (e.g., 80%, 90%) or free-text comments. These non-video features may be fused with video-derived temporal features prior to model training. Fusion may occur via concatenation, attention mechanisms, or late-fusion ensembles. This step may enable models to leverage both video dynamics (e.g., pose, SSI, SPP, etc.) and contextual information for improved accuracy and fairness. The set of non-video feature extraction steps may further include training of particular non-video features 624.

At operation 632, the system trains the model to generate a risk level prediction and/or severity score based on the video/non-video features. The system may, for example, train one or more classifier/regressor models (e.g., gradient-boosted trees, temporal transformers) to predict the risk level or severity score. Calibration (Platt or isotonic) aligns predicted probabilities with clinical ground truth. Cross-validation may improve reproducibility.

At operation 634, the system performs sensitivity validation. Performance metrics may include sensitivity, specificity, AUC, and repeatability (ICC, MDC). Models may be evaluated on held-out test sets. Results are logged with dataset/model version identifiers, acceptance criteria, and signed model cards.

For illustrative purposes, the model training module 600 is described with reference to implementations described above with respect to one or more examples described herein. Other implementations, however, are possible. In some aspects, the steps in FIG. 6 may be implemented in program code that is executed by one or more computing devices such as the object transfer task analysis system 100, the user device 120, or other system in FIG. 1. In some aspects, one or more operations shown in FIG. 6 may be omitted or performed.

### Image Analysis and Diagnosis Module

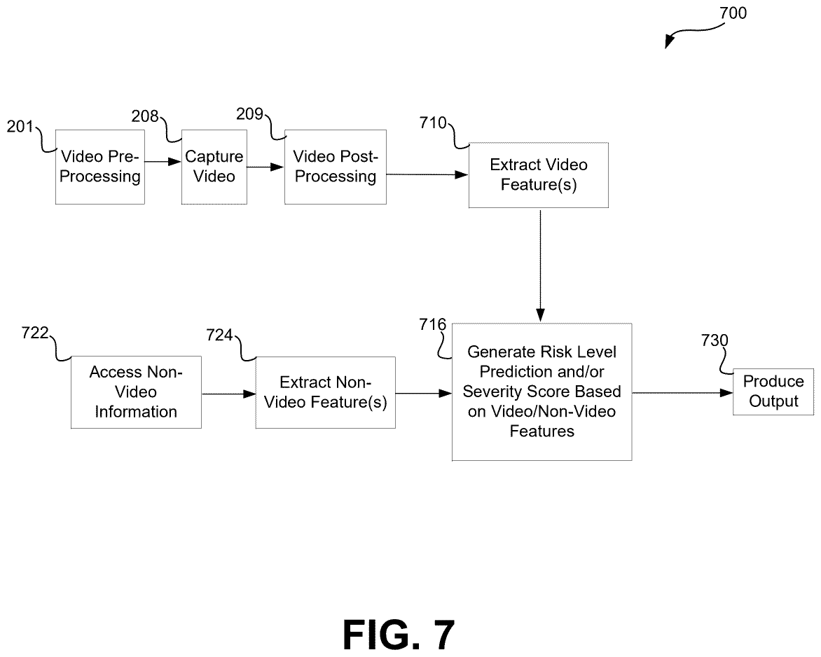

Turning now to FIG. 7, additional details are provided regarding an image analysis and diagnosis module 700 for analyzing video for use in diagnosing particular conditions. For instance, the flow diagram shown in FIG. 7 may correspond to operations executed by computing hardware found in the object transfer task analysis system 100 as it executes the image analysis and diagnosis module 700.

At operations 201, 208, and 209, the system performs a set of image capture steps(e.g., using the steps described herein with respect to the image capture module 200). At operation 722, the system accesses non-video information (e.g., about a patient) and extracts non-video features 724. This may include, for example, demographic, clinical, human input, or device metadata (e.g., age, sex, diagnosis, symptom duration, site, device type, completion rate, free-text comments), and are ingested in parallel. These may be encoded as scalars, categorical embeddings, or structured vectors.

The system then initiates a set of model prediction steps that include extracting video features 710 from the captured video. This includes, for example, extracting per-frame pose features (e.g., as described above, and extracting per-video temporal features (e.g., as described above). At operation 716, the system generates a risk level prediction and/or severity score based on the video/non-video features. The system may, for example, cause the machine learning model (e.g., the training of which is described above) to generate the risk level prediction and/or severity score. The system may be configured to log the model version and/or provenance metadata along with the prediction/severity score. At operation 730, the system may produce an output with feature attributions. In some embodiments, the output may include a diagnosis with respect to a condition, for example, based on the risk level prediction and/or severity score

For illustrative purposes, the image analysis and diagnosis module 700 is described with reference to implementations described above with respect to one or more examples described herein. Other implementations, however, are possible. In some aspects, the steps in FIG. 7 may be implemented in program code that is executed by one or more computing devices such as the object transfer task analysis system 100, the user device 120, or other system in FIG. 1. In some aspects, one or more operations shown in FIG. 7 may be omitted or performed.

### Treatment Recommendation Module

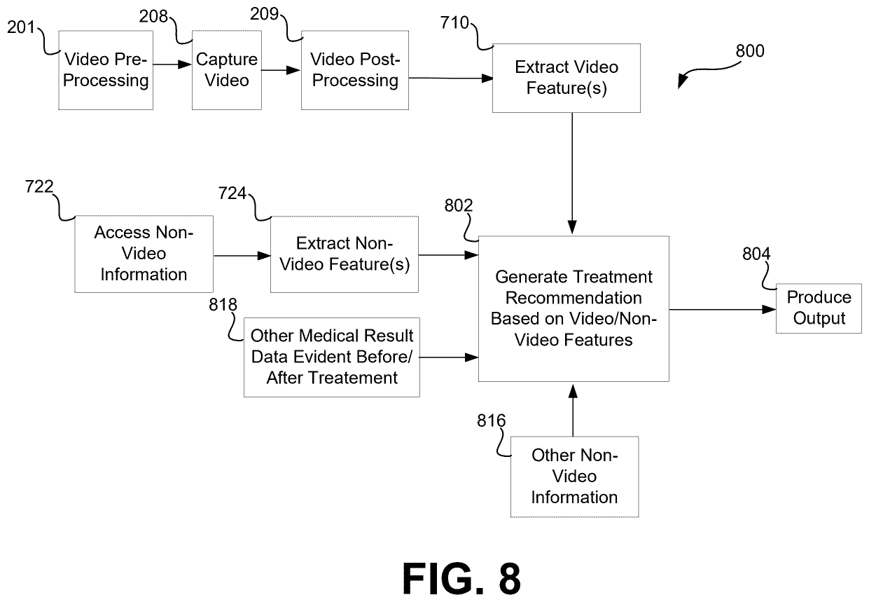

Turning now to FIG. 8, additional details are provided regarding a treatment diagnosis module 800 for generating treatment recommendations. For instance, the flow diagram shown in FIG. 8 may correspond to operations executed by computing hardware found in the object transfer task analysis system 100 as it executes the treatment recommendation module 800.

When executing the treatment diagnosis module 800 at operations 201, 208, and 209, the system performs a set of image capture steps(e.g., using the steps described herein with respect to the image capture module 200. The system further extracts video features at operation 710 (e.g., as described above), and also accesses non-video information 722 and extracts non-video features 724. In still other embodiments, the system accesses other medical result data evident before/after treatment at operation 818. In some embodiments, this may include treatment information that has already been performed for a particular individual for whom a recommended treatment is being generated. In other embodiments, the system, at operation 816, accesses other non-video information (e.g., about the individual, about one or more medical professionals available to perform a procedure, about scheduling availability for a recommended procedure, etc.).

At operation 802, the module 800 generates a treatment recommendation based on at least some of the video/non-video features, other medical data, and other non-video information. For example, in some embodiments, the system processes the risk level prediction and/or severity score using at least one of a machine-learning model or a rules-based model to generate a treatment recommendation. The treatment recommendation may take into account treatment success information for patients with similar diagnoses (e.g., severity scores, risk level predictions, etc.) with respect to particular medical conditions. The recommendation may include, for example, a recommended practitioner to perform a procedure, a procedure timing, a set of procedures to undertake in a particular order, etc. The system may further cross reference scheduling availability to recommend the most suitable practitioner to perform a recommended procedure that takes into account timing recommendation (e.g., need surgery immediately, can wait X months, etc.).

At operations 804, the system may generate an output. In some embodiments, the system may configure a user interface to include an indication of the recommendation and provide the user interface for display on a computing device.

### Progress Tracking Module

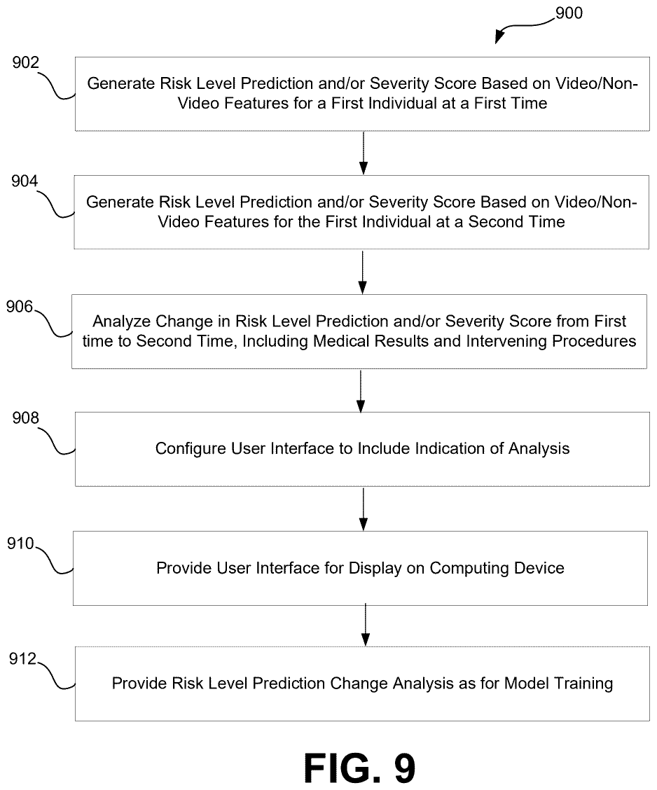

Turning now to FIG. 9, additional details are provided regarding progress tracking module for tracking changes in condition severity over time. For instance, the flow diagram shown in FIG. 9 may correspond to operations executed by computing hardware found in the object transfer task analysis system 100 as it executes the progress tracking module 900.

At operation 902, the system may generate a risk level prediction and/or severity score based on video/non-video features for a first individual at a first time (e.g., using the image analysis and diagnosis module 700). At operation 904, the system may generate a risk level prediction and/or severity score based on video-non-video features for the first individual at a second time. The system may then, at operation 906, analyze a change in risk level prediction and/or severity score form the first time to the second time. This may include analyzing procedures performed between the first time and the second time, along with information related to who performed the procedure and where. In some embodiments, the system may analyze procedure results, or derive procedure result success based on a change in the risk level or severity score from the first time to the second time. At operations 908 and 910, the system may configure a user interface to include an indication of the analysis and provide the user interface for display on a computing device. In some embodiments, the system is configured to track user progress/improvements/changes from the coin test, for example, following treatment.

In still other embodiments, at operation 912, the system is configured to provide the risk level prediction change analysis as training data for model training. For example, the system may provide video features and/or risk predictions data prior to treatment along with video features/risk prediction data after treatment as training data for a machine-learning model as discussed more below.

### Treatment Recommendation Training Module

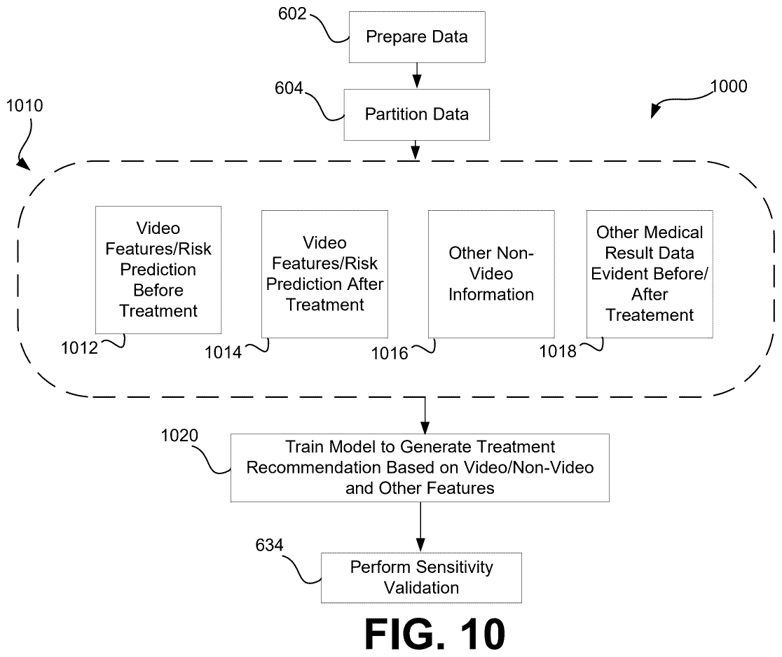

Turning now to FIG. 10, additional details are provided regarding treatment recommendation training module 1000 for training a machine-learning model for use in generating treatment plans and/or recommendations. For instance, the flow diagram shown in FIG. 10 may correspond to operations executed by computing hardware found in the object transfer task analysis system 100 as it executes the treatment recommendation training module 1000.

At operation 602, the model training module 1000 prepares the data. At operation 604, the system partitions the data into training, validation, and held-out test cohorts. The data is partitioned to maintain a balance across healthy patients and patients having a diagnosis to reduce bias during training of the machine-learning model. The system is configured to track and tag metadata to each piece of training video (e.g., site, device data, patient demographic data, procedure outcome data, and the like) to provide stratified sampling of the training data. At operation 1010, the system receives/accesses training data for the model. As shown, the training data may include risk level prediction and/or severity score data, treatment data, and outcome data (e.g., derived using any suitable process described herein). For example, the system utilize video features/risk prediction data from prior to treatment 2012 and after treatment 1014. This may, for example, in combination with other non-video information 1016 and other medical results data that are evident before/after treatment of a particular individual 1018, provide valuable training data in terms of the efficacy of particular treatment types for individuals with particular risk prediction levels and severity scores.

The system may then, at operation 1020, train the model to generate a treatment recommendation based on the video/non-video features and other features. For example, the system may train at least one of am machine-learning model or a rules-based model using the risk level prediction and/or severity score data, treatment data, and outcome data for a task of generating a treatment recommendation based on risk level prediction and/or severity score. In some embodiments, the system may perform sensitivity validation on the trained model at operation 634 as discussed above.

### Example Technical Platforms

Aspects of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example aspects, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). A machine-readable storage medium or computer-readable storage medium may include a non-transitory medium in which data can be stored and that does not include carrier waves or transitory electronic signals.

In some aspects, a non-volatile computer-readable storage medium may include a floppy disk, flexible disk, hard disk, solid-state storage (SSS) (e.g., a solid-state drive (SSD), solid state card (SSC), solid state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some aspects, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where various aspects are described to use a computer-readable storage medium, other types of computer-readable storage media may be substituted for or used in addition to the computer-readable storage media described above.

Various aspects of the present disclosure may also be implemented as methods, apparatuses, systems, computing devices, computing entities, and/or the like. As such, various aspects of the present disclosure may take the form of a data structure, apparatus, system, computing device, computing entity, and/or the like executing instructions stored on a computer-readable storage medium to perform certain steps or operations. Thus, various aspects of the present disclosure also may take the form of entirely hardware, entirely computer program product, and/or a combination of computer program product and hardware performing certain steps or operations.

Various aspects of the present disclosure are described below with reference to block diagrams and flowchart illustrations. Thus, each block of the block diagrams and flowchart illustrations may be implemented in the form of a computer program product, an entirely hardware aspect, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some examples of aspects, retrieval, loading, and/or execution may be performed in parallel such that multiple instructions are retrieved, loaded, and/or executed together. Thus, such aspects can produce specially configured machines performing the steps or operations specified in the block diagrams and flowchart illustrations. Accordingly, the block diagrams and flowchart illustrations support various combinations of aspects for performing the specified instructions, operations, or steps.

### Example System Architecture

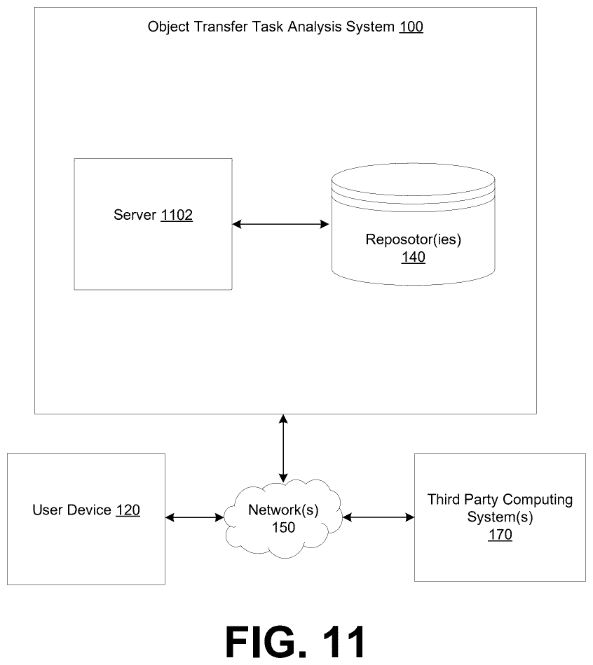

FIG. 11 is a block diagram of an example of a system architecture 1100 that can be for diagnosing and generating treatment plans for particular conditions based on object transfer task analysis as described herein. As may be understood from FIG. 11, the system architecture 1100 in some aspects may include an object transfer task analysis system 100 that comprises one or more servers 1102 and a data repository 140. The data repository 140 may be made up of computing components such as servers, routers, data storage, networks, and/or the like that are used on the object transfer task analysis system 100 to store and manage patient data, training data, and other data described herein.

As previously noted, the object transfer task analysis system 100 may provide a platform that is available more networks 150. Here, an entity may access the service via a user device 120. For example, the object transfer task analysis system 100 may provide the service through a website that is accessible to the user device 120 via the one or more networks 150, a software application on the user device 120 and the like.

Accordingly, the server(s) 1102 may execute the image capture module 200, model training module 600, image analysis and diagnosis module 700, treatment recommendation module 800, progress tracking module 900, and treatment recommendation training module 1000as described herein. Further, according to particular aspects, the server(s) 1102 may provide one or more graphical user interfaces (e.g., one or more webpages, webform, and/or the like through the website) through which users can interact with the object transfer task analysis system 100. Furthermore, the server(s) 1102 may provide one or more interfaces that allow the object transfer task analysis system 100 to communicate with third-party computing system(s) 170 such as one or more suitable application programming interfaces (APIs), direct connections, and/or the like.

### Example Computing Hardware

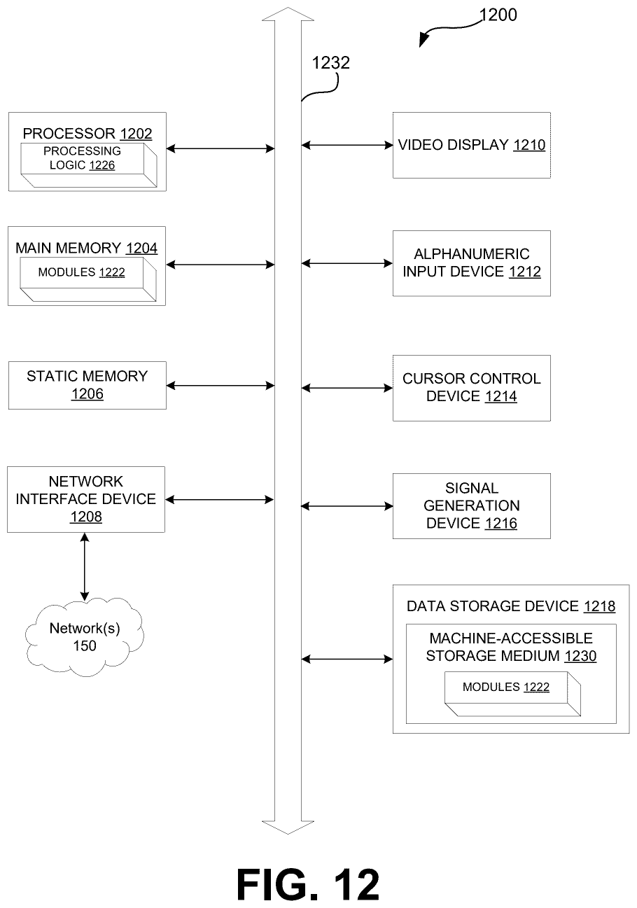

FIG. 12 illustrates a diagrammatic representation of a computing hardware device 1200 that may be used in accordance with various aspects. For example, the hardware device 1200 may be computing hardware such as a server 120 as described in FIG. 11. According to particular aspects, the hardware device 1200 may be connected (e.g., networked) to one or more other computing entities, storage devices, and/or the like via one or more networks such as, for example, a LAN, an intranet, an extranet, and/or the Internet. As noted above, the hardware device 1200 may operate in the capacity of a server and/or a client device in a client-server network environment, or as a peer computing device in a peer-to-peer (or distributed) network environment. In some aspects, the hardware device 800 may be a personal computer (PC), a tablet PC, a set-top box (STB), a Personal Digital Assistant (PDA), a mobile device (smartphone), a web appliance, a server, a network router, a switch or bridge, or any other device capable of executing a set of instructions (sequential or otherwise) that specify actions to be taken by that device. Further, while only a single hardware device 1200 is illustrated, the term “hardware device,” “computing hardware,” and/or the like shall also be taken to include any collection of computing entities that individually or jointly execute a set (or multiple sets) of instructions to perform any one or more of the methodologies discussed herein.

A hardware device 1200 includes a processor 1202, a main memory 1204 (e.g., read-only memory (ROM), flash memory, dynamic random-access memory (DRAM) such as synchronous DRAM (SDRAM), Rambus DRAM (RDRAM), and/or the like), a static memory 1206 (e.g., flash memory, static random-access memory (SRAM), and/or the like), and a data storage device 1218, that communicate with each other via a bus 1232.

The processor 1202 may represent one or more general-purpose processing devices such as a microprocessor, a central processing unit, and/or the like. According to some aspects, the processor 1202 may be a complex instruction set computing (CISC) microprocessor, reduced instruction set computing (RISC) microprocessor, very long instruction word (VLIW) microprocessor, a processor implementing other instruction sets, processors implementing a combination of instruction sets, and/or the like. According to some aspects, the processor 1202 may be one or more special-purpose processing devices such as an application specific integrated circuit (ASIC), a field programmable gate array (FPGA), a digital signal processor (DSP), network processor, and/or the like. The processor 1202 can execute processing logic 1226 for performing various operations and/or steps described herein.

The hardware device 1200 may further include a network interface device 1208, as well as a video display unit 1210 (e.g., a liquid crystal display (LCD), a cathode ray tube (CRT), and/or the like), an alphanumeric input device 1212 (e.g., a keyboard), a cursor control device 814 (e.g., a mouse, a trackpad), and/or a signal generation device 1216 (e.g., a speaker). The hardware device 1200 may further include a data storage device 1218. The data storage device 1218 may include a non-transitory computer-readable storage medium 1230 (also known as a non-transitory computer-readable storage medium or a non-transitory computer-readable medium) on which is stored one or more modules 1222 (e.g., sets of software instructions) embodying any one or more of the methodologies or functions described herein. For instance, according to particular aspects, the modules 1222 the image capture module 200, model training module 600, image analysis and diagnosis module 700, treatment recommendation module 800, progress tracking module 900, and treatment recommendation training module 1000 as described herein. The one or more modules 1222 may also reside, completely or at least partially, within main memory 804 and/or within the processor 1202 during execution thereof by the hardware device 1200-main memory 1204 and processor 1202 also constituting computer-accessible storage media. The one or more modules 1222 may further be transmitted or received over a network 150 via the network interface device 1208.

While the computer-readable storage medium 1230 is shown to be a single medium, the terms “computer-readable storage medium” and “machine-accessible storage medium” should be understood to include a single medium or multiple media (e.g., a centralized or distributed database, and/or associated caches and servers) that store the one or more sets of instructions. The term “computer-readable storage medium” should also be understood to include any medium that is capable of storing, encoding, and/or carrying a set of instructions for execution by the hardware device 1200 and that causes the hardware device 1200 to perform any one or more of the methodologies of the present disclosure. The term “computer-readable storage medium” should accordingly be understood to include, but not be limited to, solid-state memories, optical and magnetic media, and/or the like.

### System Operation

The logical operations described herein may be implemented (1) as a sequence of computer implemented acts or one or more program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, steps, structural devices, acts, or modules. These states, operations, steps, structural devices, acts, and modules may be implemented in software, in firmware, in special purpose digital logic, and any combination thereof. Greater or fewer operations may be performed than shown in the figures and described herein. These operations also may be performed in a different order than those described herein.

### Conclusion

While this specification contains many specific aspect details, these should not be construed as limitations on the scope of any invention or of what may be claimed, but rather as descriptions of features that may be specific to particular aspects of particular inventions. Certain features that are described in this specification in the context of separate aspects also may be implemented in combination in a single aspect. Conversely, various features that are described in the context of a single aspect also may be implemented in multiple aspects separately or in any suitable sub-combination. Moreover, although features may be described above as acting in certain combinations and even initially claimed as such, one or more features from a claimed combination may in some cases be excised from the combination, and the claimed combination may be a sub-combination or variation of a sub-combination.

Similarly, while operations are described in a particular order, this should not be understood as requiring that such operations be performed in the particular order described or in sequential order, or that all described operations be performed, to achieve desirable results. In certain circumstances, multitasking and parallel processing may be advantageous. Moreover, the separation of various components in the various aspects described above should not be understood as requiring such separation in all aspects, and the described program components (e.g., modules) and systems may be integrated together in a single software product or packaged into multiple software products.

Many modifications and other aspects of the disclosure will come to mind to one skilled in the art to which this disclosure pertains having the benefit of the teachings presented in the foregoing descriptions and the associated drawings. Therefore, it is to be understood that the disclosure is not to be limited to the specific aspects disclosed and that modifications and other aspects are intended to be included within the scope of the appended claims. Although specific terms are employed herein, they are used in a generic and descriptive sense only and not for the purposes of limitation.

What is claimed is:

1. A method comprising:
capturing, by computing hardware, video;
extracting, by the computing hardware, one or more per-frame pose features from the video;
extracting, by the computing hardware, one or more per-video temporal features from the video;
causing, by the computing hardware, at least one of a machine-learning model or a rules-based mode to generate at least one of a risk level prediction or a severity score based on the one or more per-frame pose features and the one or more per-video temporal features, the risk level prediction and the severity score being related to a potential diagnosis of a medical condition;
producing, by the computing hardware, an output based on at least one of the risk level prediction or the severity score;
configuring, by the computing hardware, a graphical user interface based on the output; and
providing, by the computing hardware, the graphical user interface for display on a computing device.

2. The method of claim 1, wherein:
the video comprises video of an object transfer task;
the one or more per-frame pose features comprise one or more hand or arm landmarks for each frame of the video; and
extracting the one or more per-video temporal features comprises aggregating a sequence of the one or more per-frame pose features to derive at least one of a spectral stability index during performance of the object transfer task, a stack placement precision index for the object transfer task, or a completion time for the object transfer task.

3. The method of claim 2, wherein the stack placement precision index defines a measure of how accurately each object in the object transfer task is placed relative to a second object.

4. The method of claim 2, wherein the spectral stability index provides an indication of a stability of at least a portion of a body of an individual performing the object transfer task during performance of the object transfer task.

5. The method of claim 1, wherein the output comprises a diagnosis with respect to the medical condition.

6. The method of claim 1, wherein capturing the video comprises at least one of:
calibrating, by the computing hardware, an imaging device against a set of video capture constraints;
redacting, by the computing hardware, at least one piece of identifying information from the video; or
storing, by the computing hardware, metadata in association with the video, the metadata including at least one of a device ID of the imaging device, one or more calibration parameters of the imaging device, or a configuration hash of the imaging device.

7. The method of claim 1, wherein the medical condition is cervical myelopathy.

8. A system comprising:
a non-transitory computer-readable medium storing instructions; and
a processing device communicatively coupled to the non-transitory computer-readable medium, wherein the processing device is configured to execute the instructions and thereby perform operations comprising:
preparing a set of training data, the set of training data comprising a set of videos and each video in the set of videos having a respective set of labels and including a respective object transfer task;
extracting, from each frame in each video in the set of videos, at least one respective per-frame pose feature;
extracting, from each video in the set of videos, at least one respective per-video temporal feature;
training, for a first task of generating at least one of a risk level prediction or a severity score for use in diagnosing a condition or recommending a treatment for the condition, a machine-learning model using each respective set of labels, the at least one respective feature pose for each frame in each video, and each at least one respective per-video temporal feature; and
providing the machine-learning model for use in performance of the first task.

9. The system of claim 8, wherein:
the at least one respective feature pose for each frame in each video and each at least one respective per-video temporal feature comprise a set of video-derived features; and
the operations further comprise:
extracting at least one non-video feature for each respective video in the set of videos, the at least one non-video feature defining at least one of a respective demographic of a respective subject in each respective video, a respective piece of clinical data for each respective subject, or respective structured input for each respective subject; and
fusing the at least one non-video feature with the set of video-derived features prior to training the machine-learning model.

10. The system of claim 8, wherein at least one respective label in each respective set of labels comprises one or more of: one or more calibration parameters for the respective video; a device ID used to capture each respective video; or one or more clinical labels for a subject present in each respective video.

11. The system of claim 10, wherein the one or more clinical labels comprise at least one of an age of the subject, a sex of the subject, a diagnosis for the subject, or symptom duration data for the subject.

12. The system of claim 8, wherein:
extracting the at least one respective per-video temporal feature comprises aggregating a sequence of the at least one respective per-frame pose feature for each respective video to derive at least one of a respective spectral stability index during performance of the object transfer task, a respective stack placement precision index for the respective object transfer task, or a completion time for the respective object transfer task;
the respective spectral stability index during performance of the object transfer task defines a tremor-free stability of a respective individual performing the respective object transfer task during performance or the object transfer task; and
the respective stack placement precision index for the respective object transfer task defines a measure of how accurately the respective individual places a first object during performance of the object transfer task with respect to at least one of a second object or a fiducial marker.

13. The system of claim 8, wherein the operations further comprise providing at least one of the respective spectral stability index during performance of the object transfer task, the respective stack placement precision index for the respective object transfer task, or the completion time for the respective object transfer task as training data for the machine-learning model.

14. The system of claim 8, wherein the operations further comprise:
receiving new video of an individual extracting one or more individual-specific per-frame pose features from the new video;
extracting one or more individual-specific per-video temporal features from the new video;
causing a machine-learning model to generate at least one of an individual-specific risk level prediction or an individual-specific severity score based on the individual-specific one or more per-frame pose features and individual-specific the one or more per-video temporal features;
producing an output from the machine-learning model based on at least one of the individual-specific risk level prediction or the individual-specific severity score, the output comprising at least one of an individual-specific condition diagnosis or an individual-specific treatment recommendation;
configuring, by the computing hardware, a graphical user interface based on the output; and
providing, by the computing hardware, the graphical user interface for display on a computing device.

15. The method of claim 14, wherein the operations further comprise:
providing the new video of the individual, the one or more individual-specific per-frame pose features from the new video, one or more individual-specific per-video temporal features from the new video, and the individual-specific condition diagnosis or the individual-specific treatment recommendation as additional training data to the machine learning model for the first task.

16. A method comprising:
receiving, by computing hardware, a first video at a first time;
extracting, by the computing hardware, one or more first per-frame pose features from the first video;
extracting, by the computing hardware, one or more first per-video temporal features from the first video;
causing, by the computing hardware, at least one of a first machine-learning model or a first rules-based mode to generate at least one of a first risk level prediction or a first severity score based on the one or more first per-frame pose features and the one or more first per-video temporal features, the first risk level prediction or the first severity score being related to a potential diagnosis of a medical condition;
causing, by the computing hardware, at least one of a second machine-learning model or a second rules-based model to generate a treatment recommendation based on the at least one of the first risk level prediction or the first severity score;
receiving, by computing hardware, a second video at a second time subsequent to execution of at least a portion of a treatment plan indicated by the treatment recommendation;
extracting, by the computing hardware, one or more second per-frame pose features from the second video;
extracting, by the computing hardware, one or more second per-video temporal features from the second video;
causing, by the computing hardware, at least one of the first machine-learning model or the first rules-based mode to generate at least one of a second risk level prediction or a second severity score based on the one or more second per-frame pose features and the one or more second per-video temporal features;
generating a comparison of at least one of the first risk level prediction or the second severity score with the second risk level prediction or the second severity score based;
configuring, by the computing hardware a graphical user interface with an indication of the comparison; and
providing, by the computing hardware, the graphical user interface for display on a computing device.

17. The method of claim 16, further comprising providing, by the computing hardware, the comparison and the treatment plan as training data to at least one of the second machine-learning model or the rules-based model.

18. The method of claim 16, wherein:
the first machine-learning model is the second machine-learning model; and
the first rules-based model is the second rules-based model.
